(19) United States
(12) Patent Application Publication
(10) Pub. No.: US 2014/0323348 A1
(43) Pub. Date: Oct. 30, 2014

(54) EXPRESSION OF G-PROTEIN COUPLED RECEPTORS (GPCRs)

(71) Applicant: Duke University, Durham, NC (US)

(72) Inventors: Hiroaki Matsunami, Durham, NC (US); Yue Jiang, Durham, NC (US); Ming-Shan Chien, Durham, NC (US)

(21) Appl. No.: 14/362,010

(22) PCT Filed: Nov. 30, 2012
(86) PCT No.: PCT/US12/67434

§ 371 Date: May 30, 2014

Related U.S. Application Data
(60) Provisional application No. 61/564,964, filed on Nov. 30, 2011.

(51) Int. Cl. G01N 33/74; G01N 33/50; C12N 15/85

(52) U.S. Cl. CPC G01N 33/74; C12N 15/85; G01N 33/5041
USPC 506/9; 435/375; 435/348; 435/254.2; 435/325; 435/252.33; 435/252.3; 435/252.35; 435/252.34; 435/254.21; 435/361; 435/365; 435/357; 435/369; 435/367; 435/352; 435/7.21; 435/7.31; 435/7.32; 435/6.13

(57) ABSTRACT

Disclosed are methods of promoting and/or enhancing G-Protein Coupled Receptor (GPCR) localization to the cell membrane and/or cell surface; methods of promoting and/or enhancing GPCR functional expression; and methods and assays for screening or identifying ligands (e.g., agonists or antagonists) that bind GPCRs. Also provided are vectors, recombinant cells, and stable cell lines for use in the methods and assays.

### CROSS-REFERENCE TO RELATED APPLICATIONS

This application claims priority to U.S. Provisional Patent Application No. 61/564,964, filed Nov. 30, 2011, which is incorporated herein by reference in its entirety.

### STATEMENT REGARDING FEDERALLY SPONSORED RESEARCH

This invention was made with government support under grant R01DC010857 awarded by the National Institues of Health. The government has certain rights in the invention.

### INTRODUCTION

Studies on G-protein coupled receptors (GPCRs), (e.g., functional determination and characterization, identification of ligands (agents that act as agonist or antagonist)) largely depend on successful recombinant expression of the receptors in cell culture. That is, GPCRs need to be expressed in a way that allows for proper function. However, when expressed in heterologous systems, GPCRs can be localized in the cytosol and fail to translocate to the cell surface. An example of one large family of GPCRs, the class C GPCRs, which include chemosensory receptors (e.g., vomeronasal receptors such as V2Rs and taste receptors such as T1R1 and T1R2) are difficult to express on the cell surface in heterologous expression systems. Thus, it would be beneficial to develop methods and cell lines that increase or improve the surface expression level of GPCRs that are difficult to express, and incorporate such methods and cell lines in assays that allow functional characterization of GPCRs as well as for the screening of candidate agonist/antagonist compounds. Such methods, cell lines, and assays would be useful in characterizing the binding profile of a GPCR and help to identify compounds as potential agonists and/or antagonists.

### SUMMARY

In an aspect the disclosure relates to a cell line comprising a first polynucleotide sequence encoding a GPCR and a second polynucleotide sequence encoding a Tmem30A polypeptide.

In another aspect the disclosure relates to a cell line comprising a first polynucleotide sequence encoding a GPCR and a second polynucleotide sequence encoding a Tmem30A polypeptide, wherein the cell line further comprises deletion or knock-down of a calreticulin.

In an aspect the disclosure relates to a recombinant cell comprising a first polynucleotide sequence encoding a GPCR and a second polynucleotide sequence encoding a Tmem30A polypeptide, wherein GPCR expression is localized to the cell surface.

In another aspect the disclosure relates to a recombinant cell comprising a first polynucleotide sequence encoding a GPCR and a second polynucleotide sequence encoding a Tmem30A polypeptide, wherein the cell line further comprises deletion or knock-down of a calreticulin.

In a further aspect, the disclosure relates to a cell line and/or a recombinant cell comprising a first polynucleotide sequence encoding a GPCR and a second polynucleotide sequence that encodes a Tmem30A protein having at least 80% sequence similarity to any of SEQ ID NOs: 1, 3, 5, or 7.

In yet another aspect the disclosure relates to a method for expressing a GPCR in a cell, where the method comprises providing a cell expressing a GPCR and a Tmem30A protein, and propagating, growing, culturing, or maintaining the cell under conditions effective to promote and/or increase the localization of the GPCR to the cell membrane, the cell surface, or a combination thereof. In some embodiments, the cell further includes deletion of a calreticulin. In some embodiments, the recombinant cell further expresses a protein selected from REEP, RTP1, and RTP2. In some embodiments, the GPCR is a class C GPCR. In some embodiments, the GPCR is a vomeronasal receptor or an odorant receptor.

In a further aspect the disclosure provides a method for identifying a GPCR ligand, where the method comprises providing a cell expressing a GPCR and a Tmem30A protein, propagating, growing, culturing, or maintaining the cell under conditions effective to promote and/or increase the localization of the GPCR to the cell membrane, the cell surface, or a combination thereof, contacting the cell in culture or in vitro with a candidate GPCR ligand under conditions that allow for binding of the candidate GPCR ligand to the GPCR, and detecting a signal generated by the binding of the test compound to the GPCR, wherein the candidate GPCR ligand is identified as a GPCR ligand when a signal is detected. In some embodiments, the cell further includes deletion of a calreticulin. In some embodiments, the recombinant cell further expresses a protein selected from REEP, RTP1, and RTP2. In some embodiments, the GPCR is a class C GPCR. In some embodiments, the GPCR is a vomeronasal receptor or an odorant receptor.

In still another aspect, the disclosure provides a method for enhancing functional expression of a GPCR in a cell, where the method comprises providing a cell expressing a GPCR and a Tmem30A protein, and propagating, growing, culturing, or maintaining the cell under conditions effective to promote and/or increase the localization of the GPCR to the cell membrane, the cell surface, or a combination thereof. In some embodiments, the cell further includes deletion of a calreticulin. In some embodiments, the recombinant cell further expresses a protein selected from REEP, RTP1, and RTP2. In some embodiments, the GPCR is a class C GPCR. In some embodiments, the GPCR is a vomeronasal receptor or an odorant receptor.

In an aspect the disclosure provides a method for increasing localization of a GPCR to a cell surface membrane, where the method comprises providing a cell expressing a GPCR and a Tmem30A protein, and propagating, growing, culturing, or maintaining the cell under conditions effective to promote and/or increase the localization of the GPCR to the cell surface membrane. In some embodiments, the cell further includes deletion of a calreticulin. In some embodiments, the recombinant cell further expresses a protein selected from REEP, RTP1, and RTP2. In some embodiments, the GPCR is a class C GPCR. In some embodiments, the GPCR is an vomeronasal receptor or an odorant receptor.

The disclosure provides other aspects and embodiments that will be apparent to those of skill in the art in light of the following description.

### DESCRIPTION OF THE FIGURES

FIG. 1 is a schematic diagram of the expressed receptors and co-factors in (A) the main olfactory system for odorants in mice, and in (B) the accessory olfactory system for pheromones.

FIG. 2 displays amino acid sequences for Tmem30A proteins, including human (SEQ ID NO:1); mouse (SEQ ID NO:3); C. elegans (CHAT-1, SEQ ID NO:5); and yeast (Cdc50p, SEQ ID NO:7).

FIG. 3 are images of HEK293T cells demonstrating Tmem30A promotes cell-surface expression of GPCRs.

FIG. 4 are images of cells co-transfected with Tmem30A and various tagged chemosensory and non-chemosensory GPCRs and non-GPCR transmembrane proteins. Tmem30A enhances surface expression of HAtagged T1Rs and T2Rs, slightly for one odorant receptor (OR) Rho-Olfr62, but not the other receptors and membrane proteins tested. T1R3 was co-transfected because it forms complexes with T1R1 and T1R2.

FIG. 5 are images of HEK cell line R24 cells, in which calreticulin is knocked down, to test the effect of Tmem30A and calreticulin knock down on GPCR surface expression. The combination of Tmem30A expression and calreticulin deletion further increases the surface staining of various Rho-tagged V2Rs in R24 cells.

FIG. 6 shows in situ hybridization in the mouse vomeronasal organ (VNO) coronal sections with probes specific for the mRNAs of (A) Gαₒ (positive control), (B) Tmem30A, (C) G_{α1} (negative control), (D) no probes, demonstrating that Tmem30A is expressed in the mouse VNO.

FIG. 7 are images and graphs for representative calcium imaging of RhoV2Rp1 co-expressed with Tmem30A responding to His-ESP6. (A) Images for calcium response. Left, no response. Right, response. (B) Purified His-ESP proteins stained by coomassie blue. (C)-(G) One representative set of experiments. In this case the Rho-V2Rp1, when co-expressed with Tmem30A, showed response to His-ESP6. Buffer is applied first as a negative control. Isoproteronol activates the endogenous p2-adrenergic receptor that triggers calcium response in the presence of GalS, and is used as a control for transfection efficiency.

### DETAILED DESCRIPTION

It will be understood that any numerical value recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between the lowest value and the highest value enumerated are to be considered to be expressly stated in this application.

Also, it is to be understood that the phraseology and terminology used herein is for the purpose of description and should not be regarded as limiting. The use herein of terms such as “comprising,” “including,” “having,” and variations thereof is meant to encompass the items listed thereafter and equivalents thereof as well as additional items. “Comprising” encompasses the terms “consisting of and “consisting essentially of.” The use of “consisting essentially of means that the composition or method may include additional ingredients and/or steps, but only if the additional ingredients and/or steps do not materially alter the basic and novel characteristics of the claimed composition or method.

The practice of the present invention employs, unless otherwise indicated, conventional techniques of organic chemistry, pharmacology, molecular biology (including recombinant techniques), cell biology, biochemistry, and immunology, which are within the skill of the art. Such techniques are explained fully in the literature, such as, “Molecular cloning: a laboratory manual” Second Edition (Sambrook et al., 1989); “Oligonucleotide synthesis” (M. J. Gait, ed., 1984); “Animal cell culture” (R. I. Freshney, ed., 1987); the series “Methods in enzymology” (Academic Press, Inc.); “Handbook of experimental immunology” (D. M. Weir & C. C. Blackwell, eds.); “Gene transfer vectors for mammalian cells” (J. M. Miller & M. P. Calos, eds., 1987); “Current protocols in molecular biology” (F. M. Ausubel et al., eds., 1987, and periodic updates); “PCR: the polymerase chain reaction” (Mullis et al., eds., 1994); and “Current protocols in immunology” (J. E. Coligan et al., eds., 1991), each of which is herein incorporated by reference in its entirety.

All patents publications and references cited herein are hereby incorporated by reference in their entireties, unless noted otherwise.

Co-Factors Involved in GPCR Trafficking and Signaling

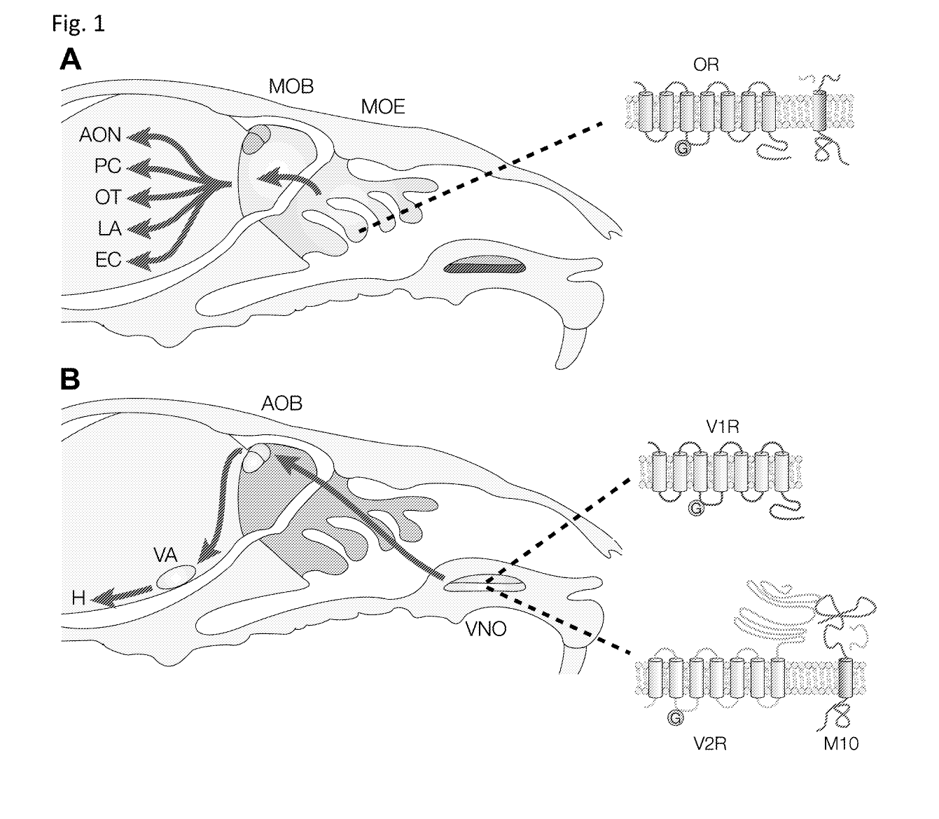

In the main olfactory system for odorants in mice (FIG. 1A), olfactory sensory neurons (OSNs) in the main olfactory epithelium (MOE) project to the main olfactory bulb (MOB) where signals are relayed to the olfactory cortex includeing the anterior olfactory nucleus (AON), the piriform cortex (PC), the olfactory tubercle (OT), the lateral part of the cortical amygdala (LA), and the entorhinal cortex (EC). The OSNs in the MOE express odorant receptors (ORs) and receptor transporting protein family (RTP). In the accessory olfactory system in mice (FIG. 1B), the vomeronasal organ (VNO) residing in the foremost region of the nasal cavity is a bone-encapsulated organ connected to the nasal cavity through a duct. The vomeronasal sensory neurons (VSNs) project to the accessory olfactory bulb (AOB) that transmits information to the vomeronasal amygdala (VA) and then hypothalamus (H), regions important for innate behaviors. VSNs in the apical layer of VNO express V1Rs. VSNs in the basal layer express V2Rs together with major histocompatibility complex (MHC) class Ib molecules M10 and β2-microglobulin (β2m).

In recent years, it has become evident that many GPCRs, such as the chemosensory receptors, do not function alone and require other components (e.g., accessory/co-factor proteins) for proper cell surface localization and signaling. Taking some chemosensory receptors as an example, the C. elegans olfactory receptor ODR-10 requires co-factors ODR-4 and UNC-101 to be trafficked to the dendritic cilia of the AWA sensory neurons. Deficiencies in odr-4 and unc-101 result in the retention of ODR10 protein in the neuron cell body and the loss of ODR-10 mediated chemotaxis behavior toward diacetyl. ODR-4 is a transmembrane protein localized to the endoplasmic reticulum (ER) and is specifically required for the function of a subset of chemosensory receptors expressed in the AWA neurons. UNC-101 encodes a μ1 subunit of the AP1 clathrin adaptor complex and is generally involved in the cilia localization of membrane proteins including receptor, channel, and transmembrane guanylyl cyclase.

In drosophila, the individual conventional ORs interact with Or83b to form heteromultimeric receptor complex to function. In Or83b mutants, dendritic localization of conventional ORs is abolished, in consistence with the loss of electrophysiological and behavioral responses to many odorants. In mammals, the taste receptor T1R1 and T1R3 interact to form the functional umami receptor when co-expressed in HEK293T cells that respond to most of the 20 standard amino acids. Similarly, T1R2 interacts with T1R3 to form the sweet receptor complex. These T1Rs, when expressed alone in a heterologous cell, fail to translocate to the cell surface and are non-functional. In addition, transient receptor potential family members PKD 1 L3 and PKD2L 1 form a candidate sour taste receptor. The interaction between these two proteins provides for the cell surface expression and the function of the receptor complex in HEK293T cells.

It has been difficult to achieve functional surface expression in heterologous cell systems for most of the mammalian olfactory receptors including ORs, V1Rs and V2Rs with the receptor transfected alone. The addition of the first 20 amino acid residues of rhodopsins to the N-terminus of ORs can increase the surface expression of some ORs. Taking these chemosensory receptor systems as model systems for the various classes of GPCRs, the inventors have identified that Tmem30A provides effective cell surface expression and functional expression of GPCRs. The methods, cells, and assays disclosed herein will provide insight on the mechanism of receptor trafficking and lead to high-throughput methods for GPCR deorphanization and identification of agents that bind to a GPCR (e.g., agonist/antagonst).

In a general sense, the disclosure relates to polynucleotides, proteins, recombinant cells, and methods for manipulating, promoting, and/or enhancing the functional expression of a GPCR in a cell wherein the polynucleotides and proteins comprise a Tmem30A sequence. The disclosure also provides assays for the identification and/or detection of an agent(s) that acts as an agonist and/or an antagonist for a functionally expressed GPCR. Surprisingly, the inventors have identified that functional expression of GPCRs can be enhanced by coexpression of the GPCR with a Tmem30A protein. In some aspects, the coexpression of a Tmem30A protein promotes or enhances the localization or trafficking of a GPCR to the cell membrane or cell surface providing for functional GPCR expression.

Described herein are compositions and methods for increasing the expression of a GPCR at the cell membrane or surface of the cell. The methods described herein incorporate nucleic acid molecules (polynucleotides, vectors, etc.) comprising a sequence that encodes a Tmem30A protein that can be incorporated into a cell and coexpressed with a GPCR. The non-limiting examples described herein demonstrate that the coexpression of a Tmem30A protein and a GPCR in a cell promotes or increases the amount of GPCR at the cell surface.

As used herein, the term “Tmem30A” when used in reference to proteins or nucleic acid refers to a Tmem30A protein or nucleic acid encoding a Tmem30A protein described herein or otherwise known or identified in the art. The term Tmem30A encompasses both proteins that are identical to a wild-type Tmem30A and those that are related to or derived from wild-type Tmem30A. Proteins and polynucleotides that are related to or derived from a Tmem30A sequence include isoforms, variants (e.g., splice variants and mutants, as well as amino acid substitutions, deletions, or additions), functional fragments (e.g., N- and C-terminal truncations, targeting domains, transmembrane domains, soluble domains), and fusion proteins. In some embodiments, Tmem30A is a wild type mammalian Tmem30A nucleic acid sequence (e.g., DNA, cDNA, RNA, mRNA) such as, for example, a sequence of SEQ ID NOs: 2, 4, 6, or 8 or a polypeptide encoded by the wild type mammalian Tmem30A nucleic acid sequence such as, for example, a sequence of SEQ ID NOs: 1, 3, 5, or 7. In some embodiments, Tmem30A is a wild type human Tmem30A nucleic acid sequence (e.g., SEQ ID NO: 2) or a polypeptide encoded by a wild type human Tmem30A nucleic acid sequence (e.g., SEQ ID NO: 1). In some embodiments, Tmem30A is a wild type murine Tmem30A nucleic acid sequence (e.g., SEQ ID NO: 4) or a polypeptide encoded by a wild type murine Tmem30A nucleic acid sequence (e.g., SEQ ID NO: 3). In some embodiments, Tmem30A is a wild type nematode CHAT-1 nucleic acid sequence (e.g., SEQ ID NO: 6) or a polypeptide encoded by a wild type nematode CHAT-1 nucleic acid sequence (e.g., SEQ ID NO: 5). In some embodiments, Tmem30A is a wild type yeast Cdc50p nucleic acid sequence (e.g., SEQ ID NO: 8) or a polypeptide encoded by a wild type yeast Cdc50p nucleic acid sequence (e.g., SEQ ID NO: 7).

The source of Tmem30A is not limited to those explicitly exemplified herein and can be derived from any organism comprising such a Tmem30A sequence/molecule. In some embodiments Tmem30A is from a eukaryotic cell (e.g., yeast, nematode, amphibian, fish, fowl, or mammal). In some embodiments, Tmem30A is from yeast (e.g., Saccharomyces). In some embodiments, Tmem30A is from a nematode (e.g., C. elegans). In some embodiments Tmem30A is from an amphibian (e.g., Xenopus). In some embodiments Tmem30A is from a fish (e.g., Danio). In some embodiments Tmem30A is from a fowl (e.g., Gallus). In some embodiments, Tmem30A is from a mammal (e.g., human, mouse, rat, chicken, cow, horse, or simian (e.g., marmoset, monkey, ape, orangutan, or chimpanzee)).

The term “G-Coupled Protein Receptor” or “GCPR” refers to any member of the large family of transmembrane receptors that typically function to bind molecules outside the cell and activate inside signal transduction pathways, ultimately inducing one or more cellular responses. G protein-coupled receptors are found only in eukaryotes, including yeast and animals. GPCRs are known to bind to a wide variety of ligands which can include light-sensitive compounds, odors, pheromones, hormones, and neurotransmitters, and vary in size from small molecules to peptides to large proteins. G protein-coupled receptors are involved in many diseases, and are also the target of approximately 40% of all modern medicinal drugs.

Binding and activation of a GPCR typically involves signal transduction pathways including the cAMP signal pathway and the phosphatidylinositol signal pathway. When a ligand binds to the GPCR it causes a conformational change in the GPCR, which allows it to act as a guanine nucleotide exchange factor (GEF). The GPCR can then activate an associated G-protein by exchanging its bound GDP for a GTP. The G-protein's α subunit, together with the bound GTP, can then dissociate from the β and γ subunits to further affect intracellular signaling proteins or target functional proteins directly depending on the a subunit type (Gαs, Gαi/o, Gαq/11, Gα12/13). Thus, binding and activation of a GPCR can be suitably detected at any step in the GPCR transduction pathway, from ligand binding to cellular response, using any technique available to one of skill in the art.

While certain classes of GPCRs lack a high degree of sequence homology, all GPCRs share a common structure and mechanism of signal transduction. Generally, GPCRs can be grouped into 6 classes based on sequence homology and functional similarity: Class A (or 1) (Rhodopsin-like), Class B (or 2) (Secretin receptor family), Class C (or 3) (Metabotropic glutamate/pheromone), Class D (or 4) (Fungal mating pheromone receptors), Class E (or 5) (Cyclic AMP receptors), Class F (or 6) (Frizzled/Smoothened). The human genome alone encodes thousands of G protein-coupled receptors, many of which are involved in detection of endogenous ligands (e.g., hormones, growth factors, etc.). Many of the GPCRs found in the human genome have unknown functions. GPCRs are involved in a wide variety of physiological processes. For example GPCRs play physiological roles in vision (opsins), sense of smell and taste (olfactory and vomeronasal receptors), mood/behavior (neurotransmitter receptors), immune response (chemokine and histamine receptors), and autonomic processes (sympathetic and parasympathetic nervous systems).

In some embodiments described herein, the GPCR is selected from any GPCR of Classes A-F. In some embodiments the GPCR is selected from a GPCR of Class C. In some embodiments the GPCR is selected from a chemosensory receptor such as, for example an odorant receptor, a taste receptor, and a vomeronasal receptor. In some embodiments the GPCR is selected from a V1R, a V2R, a T1R, and the like.

As used herein, the terms “G-Coupled Protein Receptor cell surface localization,” “GCPR cell surface localization,” “G-Coupled Protein Receptor cell surface expression,” or “GCPR cell surface expression” and equivalent terms refer to the transport or localized expression of a GCPR to a cell surface membrane. Non-limiting examples of cell surface localization include, but are not limited to, surface expression in cultured cells (see, e.g., the HEK293T cells and HEKR24 cells discussed in the Examples), localization to cilia at the tip of a dendrite, and localization to an axon terminal.

As used herein, the terms “RTP” or “REEP” refer to a RTP or a REEP protein or nucleic acid as disclosed in U.S. Pat. Nos 7,879,565, 7,838,288, 7,691,592, or 7,425,445 (incorporated herein by reference).

In one aspect the disclosure relates to a method for expressing a GPCR in a cell, where the method comprises providing a cell expressing a GPCR and a Tmem30A protein, and propagating, growing, culturing, or maintaining the cell under conditions effective to promote and/or increase the localization of the GPCR to the cell membrane and/or cell surface.

In embodiments, the cell includes a polynucleotide comprising any one or more of SEQ ID NO: 2, SEQ ID NO: 4, SEQ ID NO: 6, or SEQ ID NO: 8. In some embodiments, the polynucleotide can include additional sequences such as promoters, enhancers, or regions that encode for amino acid sequences including dimerization domains, transmembrane regions, fluorescent proteins, and the like.

The polynucleotides useful in the cells and methods disclosed herein can encode Tmem30A proteins that comprise a naturally occurring (wild-type) amino acid sequence, as well as a modified amino acid sequence that can alter, for example, the trafficking of Tmem30A to the cell membrane. Further, the polynucleotides can comprise a sequence that is codon-optimized for expression in a particular organism or cell type, while retaining the naturally-occurring sequence, or the modified amino acid sequence. Codon usage and optimization is known in the art.

Some aspects described herein relate to methods, polynucleotides, polypeptides, cells, and assays including embodiments that comprise functionally-active fragments of a Tmem30A protein. These embodiments provide an amino acid sequence that comprises less than the full length amino acid sequence of the Tmem30A protein. Such a fragment can result from a truncation at the amino terminus, a truncation at the carboxy terminus, and/or an internal deletion of one or more amino acid residues from the amino acid sequence(s). Naturally occurring fragments may result from alternative RNA splicing, from in vivo processing such as removal of the leader peptide and propeptide, and/or from protease activity. The fragments can be tested for activity by identifying function (e.g., GPCR surface expression/staining, GPCR signaling activity, or both). Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule. For example, these terms encompass functional equivalents such as, for example, fragments, N- and C-terminal truncations, extracellular domains, soluble domains, extracellular domains and/or soluble domains tethered to one or more transmembrane domains, ligand-binding domains, cell-surface binding domains, naturally occurring and/or synthetically derived (e.g., engineered) mutant sequences, variants, derivatives, orthologs, and the like.

In some embodiments, the disclosure provides a polynucleotide comprising a sequence that is at least 80 percent identical to the nucleotide sequence encoding a wild-type Tmem30A protein, or comprises a nucleotide sequence encoding polypeptides that are at least 80 percent identical to a wild-type Tmem30A. Accordingly, the nucleotide sequences can be at least 80, 81, 82, 83, 84, 85, 86, 87, 88, 89, 90, 91, 92, 93, 94, 95, 96, 97, 98, 99, or 100 percent identical to any nucleotide sequence encoding a wild type Tmem30A protein, or the nucleotide sequences can encode polypeptides that are at least 80 percent (80, 81, 82, 83, 84, 85, 86, 87, 88, 89, 90, 91, 92, 93, 94, 95, 96, 97, 98, 99, or 100 percent) identical to the wild-type Tmem30A protein. Nucleic acid molecules also include fragments of the above nucleic acid molecules which are at least about 10 contiguous nucleotides, or about 15, or about 20, or about 25, or about 50, or about 75, or about 100, or greater than about 100 contiguous nucleotides. Related nucleic acid molecules also include fragments of the above Tmem30A polynucleotide molecules which encode an amino acid sequence of a Tmem30A protein of at least about 25 amino acid residues, or about 50, or about 75, or about 100, or greater than about 100 amino acid residues of the wild type protein. The isolated nucleic acid molecules include those molecules which comprise nucleotide sequences which hybridize under moderate or highly stringent conditions as defined below with any of the above nucleic acid molecules. In embodiments, the nucleic acid molecules comprise sequences which hybridize under moderate or highly stringent conditions with a nucleic acid molecule encoding a polypeptide, which polypeptide comprises a sequence as shown in any of SEQ ID NO:1, SEQ ID NO:3, SEQ ID NO:5, or SEQ ID NO:7, or with a nucleic acid fragment as defined above, or with a nucleic acid fragment encoding a polypeptide as defined above. It is also understood that related nucleic acid molecules include sequences which are complementary to any of the above nucleotide sequences.

The term “high stringency conditions” refers to those conditions that (1) employ low ionic strength reagents and high temperature for washing, for example, 0.015 M NaCl/0.0015 M sodium citrate/0.1% NaDodSO₄ (SDS) at 50° C., or (2) employ during hybridization a denaturing agent such as formamide, for example, 50% (vol/vol) formamide with 0.1% bovine serum albumin/0.1%. Alternatively, Fico11/0.1% polyvinylpyrrolidone/50 mM sodium phosphate buffer at pH 6.5 may be used with 750 mm NaC1, 75 mm sodium citrate at 42° C. Another example is the use of 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/mL), 0.1% SDS, and 10% dextran sulfate at 42° C., with washes at 42° C. in 0.2×SSC and 0.1% SDS.

The term “moderate stringency conditions” refers to conditions which generally include the use of a washing solution and hybridization conditions (e.g., temperature, ionic strength, and percent SDS) less stringent than described above. A non-limiting example of moderately stringent conditions includes overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 μL/mL denatured sheared salmon sperm DNA, followed by washing the filters in 1× SSC at about 37-50° C. Those skilled in the art will recognize how to adjust the temperature, ionic strength and other parameters as necessary in order to accommodate factors such as nucleic acid length and the like.

Relatedness of Nucleic Acid Molecules and/or Amino Acid Sequences

The term “identity” refers to a relationship between the sequences of two or more amino acid sequences or two or more nucleic acid molecules, as determined by comparing the sequences. In the art, “identity” also means the degree of sequence relatedness between amino acid or nucleic acid molecule sequences, as the case may be, as determined by the match between strings of nucleotide or amino acid sequences. “Identity” measures the percent of identical matches between two or more sequences with gap alignments addressed by a particular mathematical model or computer programs (i.e., “algorithms”).

The term “similarity” is a related concept, but in contrast to “identity”, refers to a measure of similarity which includes both identical matches and conservative substitution matches. Since conservative substitutions apply to polypeptides and not nucleic acid molecules, similarity only deals with polypeptide sequence comparisons. If two polypeptide sequences have, for example, 10/20 identical amino acids, and the remainder are all non-conservative substitutions, then the percent identity and similarity would both be 50%. If in the same example, there are 5 more positions where there are conservative substitutions, then the percent identity remains 50%, but the percent similarity would be 75% (15/20). Therefore, in cases where there are conservative substitutions, the degree of similarity between two polypeptide sequences will be higher than the percent identity between those two sequences.

Identity and similarity of related nucleic acid molecules and polypeptides can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 19933; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M. Stockton Press, New York, 1991; and Carillo, H., and Lipman, D., SIAM J. Applied Math., 48:1073 (1988).

Non-limiting methods for determining identity and/or similarity are designed to give the largest match between the sequences tested. Methods to determine identity and similarity are codified in publicly available computer programs and are well known in the art. Preferred computer program methods to determine identity and similarity between two sequences include, but are not limited to, the GCG program package, including GAP (Devereux, et al., Nucleic Acids Research 12:387 [1984]; Genetics Computer Group, University of Wisconsin, Madison, Wis.), BLASTP, BLASTN, and FASTA (Atschul et al., J. Molec. Biol. 215:403-410 [1990]). The BLAST X program is publicly available from the National Center for Biotechnology Information (NCBI) and other sources (BLAST Manual, Altschul et al., NCB NLM NIH Bethesda, Md. 20894; Altschul et al., J. Mol. Biol. 215:403-410 [1990]). The well known Smith Waterman algorithm may also be used to determine identity.

Other exemplary algorithms, gap opening penalties, gap extension penalties, comparison matrices, thresholds of similarity, etc. can be used by those of skill in the art. The particular choices to be made will depend on the specific comparison to be made, such as DNA to DNA, protein to protein, protein to DNA; and additionally, whether the comparison is between given pairs of sequences (in which case GAP or BestFit are generally preferred) or between one sequence and a large database of sequences (in which case FASTA or BLASTA are preferred).

Vectors

The polynucleotides useful in the various aspects described herein may be employed for expressing polypeptides in cells by recombinant techniques. Thus, for example, the polynucleotide may be included in any one of a variety of expression vectors for expressing a polypeptide. In some embodiments of the present invention, vectors include, but are not limited to, chromosomal, nonchromosomal and synthetic DNA sequences (e.g., derivatives of SV40, bacterial plasmids, phage DNA; baculovirus, yeast plasmids, vectors derived from combinations of plasmids and phage DNA, and viral DNA such as vaccinia, adenovirus, fowl pox virus, and pseudorabies). It is contemplated that any vector may be used as long as it is replicable and viable in the host.

In particular, some embodiments relate to recombinant constructs comprising one or more of the sequences as described above (e.g., SEQ ID NOs: 2, 4, 6, or 8, or sequences at least 80% identical thereto) and optionally a GPCR. In some embodiments, the constructs comprise a vector, such as a plasmid or viral vector, into which one or more sequences has been inserted, in a forward or reverse orientation. In still other embodiments, the heterologous structural sequence (e.g., SEQ ID NOs: 2, 4, 6, or 8, or sequences at least 80% identical thereto) is assembled in appropriate phase with translation initiation and termination sequences. In some embodiments of the present invention, the appropriate DNA sequence is inserted into the vector using any of a variety of procedures. In general, the DNA sequence is inserted into an appropriate restriction endonuclease site(s) by procedures known in the art.

Large numbers of suitable vectors are known to those of skill in the art, and are commercially available. Any other plasmid or vector may be used as long as they are replicable and viable in a recombinant/host cell. In some embodiments, mammalian expression vectors comprise an origin of replication, a suitable promoter and enhancer, and also any necessary ribosome binding sites, polyadenylation sites, splice donor and acceptor sites, transcriptional termination sequences, and 5′ flanking non-transcribed sequences. In further embodiments, recombinant expression vectors include origins of replication and selectable markers permitting transformation of the host cell (e.g., dihydrofolate reductase or neomycin resistance for eukaryotic cell culture, or tetracycline or ampicillin resistance in E. coli). The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

Embodiments provide nucleic acid constructs in the form of plasmids, vectors, transcription or expression cassettes which comprise at least one polynucleotide encoding a Tmem30A protein or a functional fragment thereof, and a suitable promoter region. Suitable vectors can be chosen or constructed, which contain appropriate regulatory sequences, such as promoter sequences, terminator sequences, polyadenylation sequences, enhancer sequences, marker genes and other sequences as desired. Vectors can be plasmids, phage (e.g. phage, or phagemid) or viral (e.g. lentivirus, adenovirus, AAV) or any other appropriate vector. In embodiments, the vector can be an expression vector (or expression constructs) for driving expression of the polynucleotide and the protein it encodes in a target cell. Vectors and methods for inserting them into a target cell are known in the art. For further details see, for example, Molecular Cloning: a Laboratory Manual: 2nd edition, Sambrook et al., 1989, Cold Spring Harbor Laboratory Press (incorporated herein by reference).

Host Cells, Recombinant Cells, Cell Lines

In aspects, the disclosure provides host (i.e., recombinant) cells containing the above-described vector constructs and/or polynucleotide sequences. In some embodiments, the host cell is a higher eukaryotic cell (e.g., a mammalian or insect cell). In other embodiments, the host cell is a lower eukaryotic cell (e.g., a yeast cell). In still other embodiments, the host cell can be a prokaryotic cell (e.g., a bacterial cell). Host cells can include, for example, Escherichia coli, Salmonella typhimurium, Bacillus subtilis, and various species within the genera Pseudomonas, Streptomyces, and Staphylococcus, as well as Saccharomycees cerivisiae, Schizosaccharomycees pombe, Drosophila S2 cells, Spodoptera Sf9 cells, Chinese hamster ovary (CHO) cells, COS-7 lines of monkey kidney fibroblasts, C127, 3T3, HEK293, HEK293T, R24, HeLa, and BHK cell lines.

Genes and the proteins genes encode can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., Molecular Cloning: A Laboratory Manual, Second Edition, Cold Spring Harbor, N.Y., [1989].

In some embodiments, this aspect relates to a cell line (e.g., heterologous 293T cell line) comprising expression of GPCR (e.g., a Class C GPCR, a vomeronasal receptor, an odorant receptor, a taste receptor) localized to the cell surface, a Tmem30A, and G_{αolf}. In some embodiments, the GPCR can be tagged with a reporting agent as are known in the art (e.g., glutathione-S-transferase (GST), c-myc, 6-histidine (6×-His), green fluorescent protein (GFP), maltose binding protein (MBP), influenza A virus haemagglutinin (HA), β-galactosidase, and GAL4). In some embodiments, the cell lines are used in the identification and/or classification of a GPCRs functional expression (e.g., ligand specificity).

In an aspect, the disclosure provides recombinant cells that comprise a GPCR and the polynucleotides described herein. In a related aspect the disclosure provides a stable cell line that comprises a GPCR and the polynucleotides described herein. In some embodiments the recombinant cell and/or the cell line further comprises a calreticulin deletion or knock-down (e.g., as the R24 cells described in the Examples). Techniques for generating (e.g., transfection) and maintaining recombinant cells are known in the art, such as those described in Sambrook et al., 1989.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Transfection can be either transient or stable. Stable transfection refers to the introduction and integration of foreign DNA into the genome of the transfected cell. Suitably a cell line or recombinant cell refers to a cell that has stably integrated foreign DNA into the genomic DNA.

The term “test compound” or “candidate compound” refers to any chemical entity, pharmaceutical, drug, and the like that can be screened for its potential binding activity to one or more GPCRs. In some embodiments such compounds may bind a GPCR and modulate the activity of the GPCR. In some embodiments the binding of the compound to the GPCR will inhibit activity of the GPCR (antagonist activity). In some embodiments the binding of the compound to the GPCR will induce or increase activity of the GPCR (agonist activity). In some embodiments test compounds identified as a GPCR ligand can be formulated and used to treat or prevent a disease, illness, sickness, or disorder of bodily function, or otherwise alter the physiological or cellular status of a sample. Therefore, test compounds comprise both known and potential therapeutic compounds. A test compound can be determined to be therapeutic by screening using the screening methods as described herein.

As used herein, the term “response,” when used in reference to an assay, refers to the generation of a detectable signal (e.g., accumulation of reporter protein, increase in ion concentration, accumulation of a detectable chemical product).

Identification of GPCR Ligands

In an aspect the disclosure provides for methods for identifying ligands that have binding activity for a GPCR. In embodiments, the method comprises providing a cell (e.g., heterologous 293T cell line) expressing a GPCR of interest (e.g., any human GPCR) and a Tmem30A protein, and G_{αolf}. Activation of a GPCR receptor results in an increase in cAMP. As such, in some embodiments, the cell line further comprises a cAMP responsive element linked with a reporting agent (e.g., luciferase) for detecting GPCR activation. A candidate compound is exposed to (contacted or administered) to the cell line. If the candidate compound is a ligand having binding activity for the GPCR, luciferase expression or a change in luciferase expression is detectable.

In some embodiments, the disclosure provides methods of screening compounds for the ability to alter GPCR activity mediated by natural ligands (e.g., identified using the methods described above). Such compounds find use in the treatment of disease mediated by GPCRs.

The disclosure contemplates the use of cell lines expressing a GPCR and a Tmem30A in assays for screening compounds for GPCR binding activity, and in particular to high throughput screening of compounds from combinatorial libraries (e.g., libraries containing greater than 10⁴ compounds). The cell lines of the present invention can be used in a variety of screening methods. In some embodiments, the cells can be used in an assay that monitors signal transduction following activation of a GPCR receptor. In other embodiments, the cells can be used in reporter gene assays that monitor cellular responses at the transcription/translation level.

In some embodiments, the assays comprise the host cells described above and are then contacted or treated with a compound or plurality of compounds (e.g., from a combinatorial library) and assayed for the presence or absence of a response. It is contemplated that at least some of the compounds in the combinatorial library can serve as agonists, antagonists, activators, or inhibitors of the GPCRs localized at the cell membrane. It is also contemplated that at least some of the compounds in the combinatorial library can serve as agonists, antagonists, activators, or inhibitors of protein acting upstream or downstream of the GPCR in a signal transduction pathway.

In some embodiments, the assays measure fluorescent signals from reporter molecules that respond to intracellular changes (e.g., Ca² concentration, membrane potential, pH, cAMP, arachidonic acid release) due to stimulation of GPCRs and/or ion channels (e.g., ligand gated ion channels; see Denyer et al., Drug Discov. Today 3:323 [1998]; and Gonzales et al., Drug. Discov. Today 4:431-39 [1999]). Examples of reporter molecules include, but are not limited to, FRET (florescence resonance energy transfer) systems (e.g., Cuo-lipids and oxonols, EDAN/DABCYL), calcium sensitive indicators (e.g., Fluo-3, FURA 2, INDO 1, and FLUO3/AM, BAPTA AM), chloride-sensitive indicators (e.g., SPQ, SPA), potassium-sensitive indicators (e.g., PBFI), sodium-sensitive indicators (e.g., SBFI), and pH sensitive indicators (e.g., BCECF).

Suitably, the host cells can be loaded with the indicator prior to exposure to the compound. Responses of the cells to treatment with the compounds can be detected by any methods known in the art, including, but not limited to, fluorescence microscopy, confocal microscopy (e.g., FCS systems), flow cytometry, microfluidic devices, FLIPR systems, and plate-reading systems. In some preferred embodiments, the response (e.g., increase in fluorescent intensity) caused by compound of unknown activity is compared to the response generated by a known agonist and expressed as a percentage of the maximal response of the known agonist. The maximum response caused by a known agonist is defined as a 100% response. Likewise, the maximal response recorded after addition of an agonist to a sample containing a known or test antagonist is detectably lower than the 100% response.

Therapeutic Agents & Pharmaceutical Compositions

The disclosure also provides aspects that relate to novel agents (or known agents having novel GPCR binding activity) identified by the methods and screening assays described herein. Accordingly, embodiments of this aspect relate to the use of an agent identified as described herein (e.g., a GPCR ligand, agonist, or antagonist) in an appropriate animal model of a disorder or disease relating to GPCR activity in order to determine the efficacy, toxicity, side effects, or mechanism of action, of treatment with such an agent.

The GPCR binding agents identified by the methods and assays described herein can be formulated as a pharmaceutical composition either alone or in combination with at least one other agent, such as a stabilizing compound, and may be administered in any sterile, biocompatible pharmaceutical carrier, including, but not limited to, saline, buffered saline, dextrose, and water.

Depending on the condition being treated, these pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration.

Pharmaceutical compositions suitable for use in the present invention include compositions wherein the active ingredients are contained in an effective amount to achieve the intended purpose. Determination of effective amounts is well within the capability of those skilled in the art.

A therapeutically effective dose refers to the amount of an active agent that ameliorates symptoms of the disease state. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. It follows that active agents having large therapeutic indices are desireable. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors which may be taken into account include the severity of the disease state; age, weight, and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation. Normal dosage amounts may vary from 0.01 to 100,000 micrograms, up to a total dose of about 1 g, depending upon the route of administration. Guidance as to particular dosages and methods of delivery is provided in the literature (See, U.S. Pat. Nos. 4,657,760; 5,206,344; or 5,225,212, all of which are herein incorporated by reference).

In addition to the active ingredients these pharmaceutical compositions may contain suitable pharmaceutically acceptable carriers comprising excipients and auxiliaries that facilitate processing of the active compounds into preparations that can be used pharmaceutically.

Pharmaceutical compositions may be manufactured in a manner that is itself known (e.g., by means of conventional mixing, dissolving, granulating, dragee-making, levigating, emulsifying, encapsulating, entrapping or lyophilizing processes).

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms.

The following Examples are provided merely for purposes of illustrating certain aspects and embodiments of the disclosure described above and should not be interpreted as limiting the scope of the appended claims.

### EXAMPLES

### Example 1

### Materials and Methods

Cloning. Coding regions of the mouse V2Rs were amplified from VNO cDNA library with Phusion® high fidelity DNA Polymerase (New England BioLabs, Inc., Ipswich, Mass.). Expression vectors for the V2Rs were constructed by cloning regions corresponding putative mature proteins into a pCI vector (Promega, Madison, Wis.) containing a 5HT receptor signal sequence and a Rhodopsin tag.

For receptor chimeras, a two step PCR method with primers corresponding to joint regions and the vector primers were used. For point mutants, primers corresponding to the mutated residue(s) were used to amplify mutants.

Cell culture. Cells were cultured in minimal essential medium (Sigma-Aldrich, St. Louis, Mo.), supplemented with 10% fetal bovine serum (Sigma-Aldrich) by volume, with GIBCO™ Penicillin-Streptomycin (10 μg/mL; Invitrogen, Carlsbad, Calif.) and FUNGIZONE™ (0.25 μg/mL, Sigma-Aldrich,), in a 37° C. incubator containing 5% CO₂.

Live cell surface staining. HEK293T cells were seeded in a 35 mm dish (BD Falcon™ Becton, Dickinson & Co., Franklin Lakes, N.J.) containing a piece of cover glass coated with poly-D-Iysine (Sigma) 24 hours prior to transfection in Minimum Essential Medium containing 10% FBS (M10). Plasmid DNA was transfected using Lipofectamine2000 (Invitrogen) together with Green fluorescent protein (GFP) as a control for transfection efficiency. Cells were stained between 24 hours to 48 hours post-transfection by incubating on ice with M10 containing 1/100 dilution of primary (anti-Rho) followed by 1/100 dilution of secondary (anti mouse Cy3) antibody, 30-45 minutes each (three washes per slide in wash buffer). Cells were fixed with 4% paraformaldehyde (PFA), mounted with Mowiol mounting medium, and observed for fluorescence.

Permiablized cell staining. Cell culture and transfection methods were the same as those described for live cell surface staining Post-transfection (24 hours to 48 hours), cells were fixed with 4% PFA in PBS for 15 min at 4° C., and then permeablized with methanol for 1 min on ice. After two washes in phosphate buffered saline (PBS), slides were blocked with blocking solution (5% skim milk in PBS) for 30 min at room temperature. Cells were stained by incubating at room temperature with blocking solution containing 1/100 dilution of primary (anti-Rho) followed by 1/100 dilution of secondary (anti mouse Cy3) antibody, 30-45 minutes each (three washes per slide in wash buffer). Slides were mounted with Mowiol mounting medium and observed for fluorescence.

Ligand protein preparation. ORFs encoding the exocrine gland secreting peptides (ESPs) were amplified from cDNAs of mouse glands and cloned into the bacterial expression vector pET28a (Novagen). After induction with IPTG and subsequent incubation, bacteria were harvested and lysed by three cycles of freeze—thawing at −80° C. and room temperature. Pellets were resuspended in lysis buffer and subjected to sonication. His-tagged proteins were purified by standard protocols using Ni—NTA beads (Novagen). The purity of the recombinant proteins was assessed by SDS/PAGE followed by Coomasie-blue staining

Calcium imaging. After transfection (24-36 hours), cells were loaded with the calcium-sensitive dyes Fluo-4 and Fura-red for 45 min. Leica confocal microscope (excitation 488 nm, emission 500-560 nm for Fluo-4, 605-700 nm for Fura-red) was used for data acquisition. Data was collected at 3-sec interval in the live imaging mode of Leica confocal software. Cells were exposed to constant flow of bath solution (Hank's buffer containing 10 mM HEPES, 5 mM glucose, Invitrogen).

### Example 2

### Identification of Tmem30A as a GPCR Expression Co-Factor

A methodology was used to screen for co-factors involved in OR trafficking from the genes highly expressed in OSNs based on single OSN SAGE (serial analysis of gene expression) data. Previously, this expression screen successfully identified receptor transporting proteins RTP1 and RTP2, as well as the receptor expression enhancing protein REEP 1. These proteins have been used in recombinant heterologous cells (HEK293T cells) to promote the surface expression of odorant receptors. However these proteins were not effective in promoting the surface expression of certain chemosensory GPCRs such as, for example, V1Rs and V2Rs. Using this heterologous system, 340 OR-ligand interactions, with 62 ORs matched with at least one odorant in mouse and human, have been identified.

Until this work there has been no previous report that identifies any co-factors that have the ability to efficiently target functional V2Rs to cell surface in a heterologous expression system.

Tmem30A

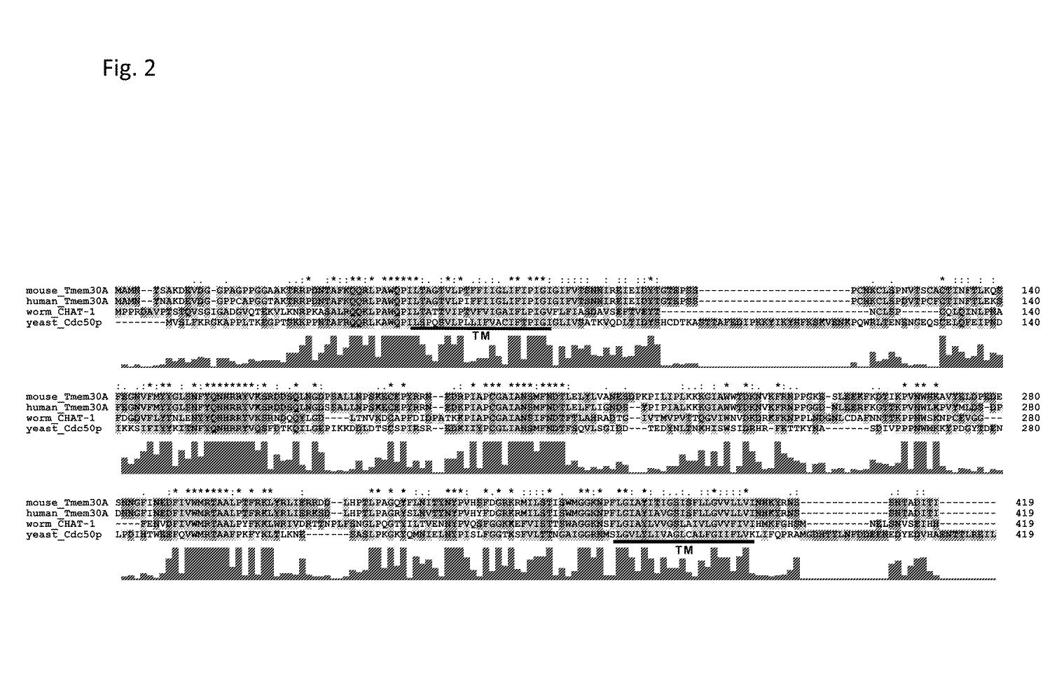

Tmem30A is a membrane protein that is highly expressed in the vomeronasal sensory neurons. Tmem30A is a well-conserved protein with homologs in yeast, worm, fly, mouse, and human (FIG. 2). The predicted topology of Tmem30A indicates it has two transmembrane domains, with the N- and C-terminal regions inside the cell. It is known to form complexes with P-type ATPase subfamily IV (P4 Atpase) members to regulate phosphatidyl serine (PS) asymmetry on biomembranes. Through translocating PS, this complex regulates membrane dynamics and participates in the intracellular vesicle trafficking The yeast and worm homologs of Tmem30A, CDC50 and CHAT-1, are also implicated as P4 ATPase chaperones that are required for proper function. Structurally, the N-terminal glycosylation sites, transmembrane regions, and the extracellular domain of Tmem30A all have a role in formation of complexes with the target ATPases.

Tmem30A Translocates GPCRs to Cell Surface

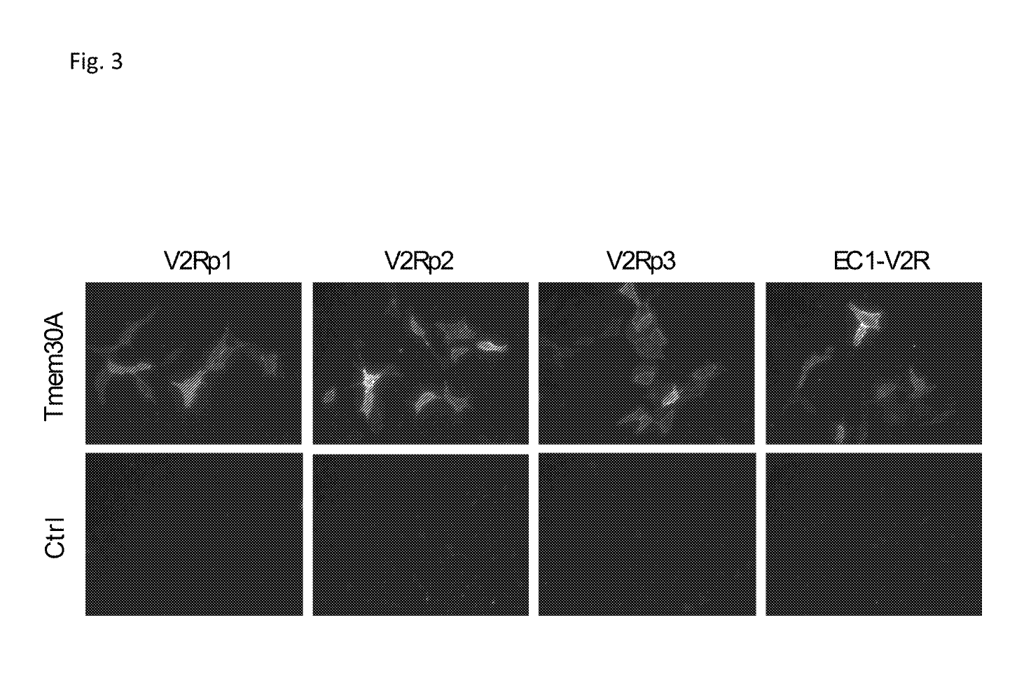

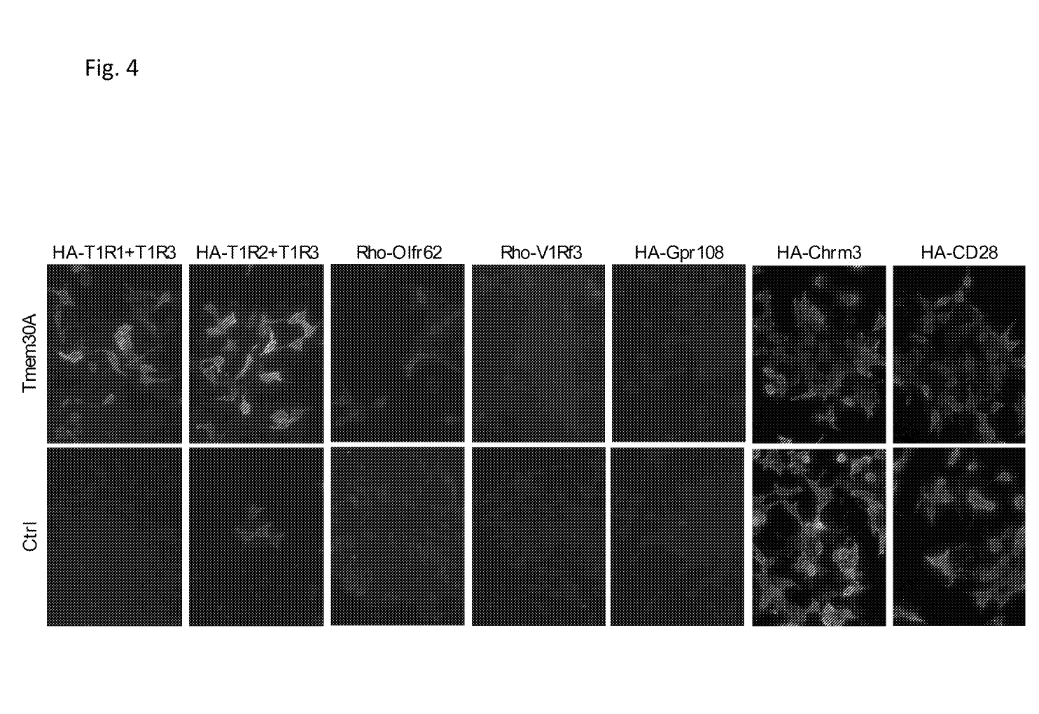

Tmem30A was identified as a likely co-factor that is expressed in the VSNs and that may be participating in the trafficking of GPCRs (vomeronasal receptors). In order to identify such co-factors, about 100 genes were selected that are highly expressed in VSNs based on the single chemosensory neuron expression profiles. The main focus were genes encoding transmembrane proteins because most of the known co-factors for chemosensory receptors contain transmembrane domains but have also included about 20 genes coding for cytosolic proteins. The cDNAs for these genes were amplified from the mouse VNO cDNA library with Phusion® high fidelity DNA Polymerase (New England BioLabs, Inc., Ipswich, Mass.) and cloned them into the pCI expression vector (Promega, Madison, Wis.). These cDNA clones are co-transfected with V1Rf3 or V2Rp1 both containing a Rho-tag at the N-terminus, which enables the evaluation of the receptor surface expression by non-permeablized immunostaining When expressed alone, V1Rf3 and V2Rp1 show poor surface expression. However, the co-expression of Tmem30A dramatically increased the surface staining of V2Rp1 but not V1Rf3 (FIG. 3). In addition, Tmem30A also promoted the surface expression of other V2Rs tested in HEK293T cells. It was next examined whether this surface trafficking effect of Tmem30A was specific for V2R or if it also works for other GPCRs and transmembrane proteins. To address this question, Tmem30A was co-transfected with various tagged chemosensory (T1R1, T1R2, O1fr62, V1Rf3) and non-chemosensory GPCRs (Chrm3, Gpr108), as well as non-GPCR transmembrane proteins (CD28). Tmem30A increased the surface expression of the taste receptors T1R1 and T1R2. Similar as V2Rs, the T1Rs are also class C GPCRs with long N-terminal regions. Among the other membrane proteins tested, Tmem30A showed a much milder enhancement. Under these conditions, Tmem30A increased the surface expression of V1Rs, non-chemosensory GPCR Gpr108 and muscarinic receptor Chrm3, or non-GPCR transmembrane protein CD28 (some of which are already surface expressed) to a lesser extent that it did V2Rs (FIG. 4).

Calreticulin Knockdown Enhances GPCR Surface Expression

In terms of surface expression, HEK293T cells were constructed that are depleted in calreticulin (see, e.g., Dey, S., and Matsunami H., “Calreticulin chaperones regulate functional expression of vomeronasal type 2 pheromone receptors.” Proc Natl Acad Sci USA. 2011 Oct. 4; 108(40):16651-6; incorporated herein by reference), which reduces the ER retention of V2Rs and increases the amount of receptors on the plasma membrane. In addition, one M10 family member M10.4 also promotes V2R surface expression in the calreticulin knock-down cells. Using this strategy, two V2Rs were matched with their ligands (V2Rp1 detecting ESPS and ESP6; and V2Rp2 detecting ESP6). As calreticulin is a common endoplasmic reticulum chaperone that controls the folding and trafficking of proteins in the ER lumen, it is involved in the normal function of HEK cells.

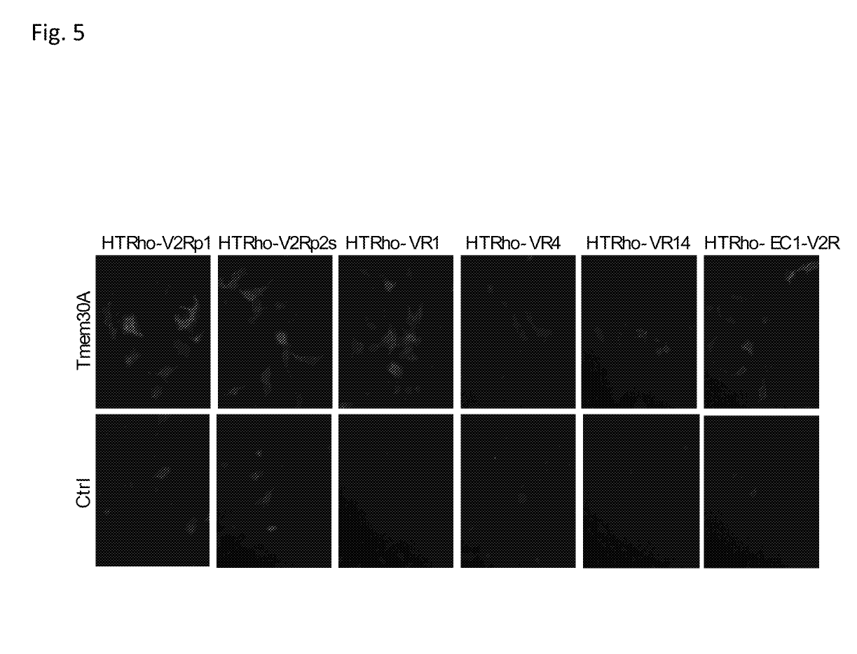

Thus, reducing the expression of calreticulin in HEK293T cells facilitates the heterologously expressed V2Rs to exit from ER and traffic to the cell surface. Using the stable calreticulin knocking down HEK cell line R24, it was tested whether Tmem30A works additively or synergistically with the calreticulin deficiency in terms of V2R surface expression. The staining showed that Tmem30A further promoted the surface trafficking in calreticulin knock down background (FIG. 5).

Tmem30A is Highly Expressed in the Mouse VSNs.

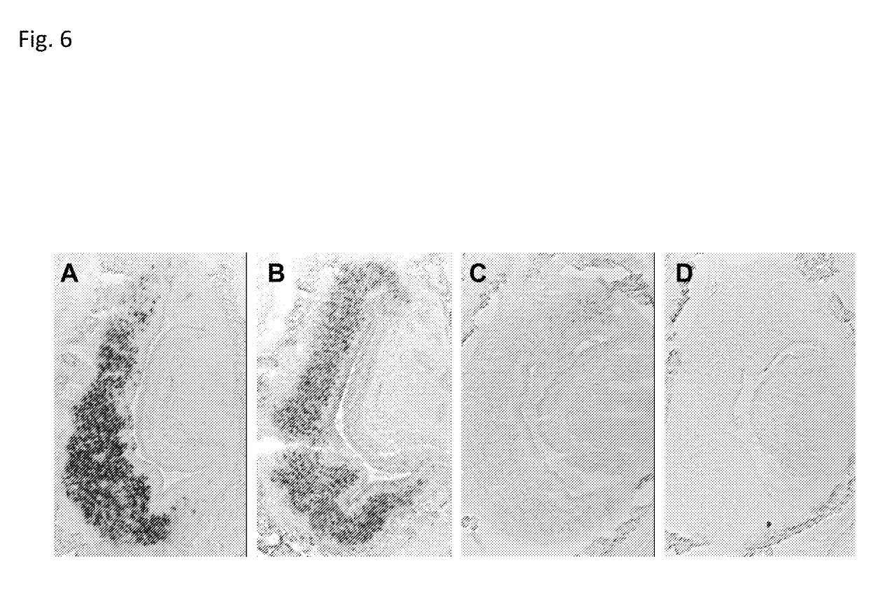

To confirm whether Tmem30A was indeed expressed in the VNO as appeared in the single cell expression profile, in situ hybridization was performed with probes specific for Tmem30A mRNA (e.g., that hybridize under assay conditions to Tmem30A mRNA sequence(s)) in the coronal sections of mouse VNO. In accordance with the expression profile, Tmem30A showed strong in situ signals in the VNO and the expression pattern was similar as Gao, the marker for V2R+VSNs (FIG. 6).

Preliminary Examination of V2Rp1 Activity in the Presence of Tmem30A in HEK293T Cells

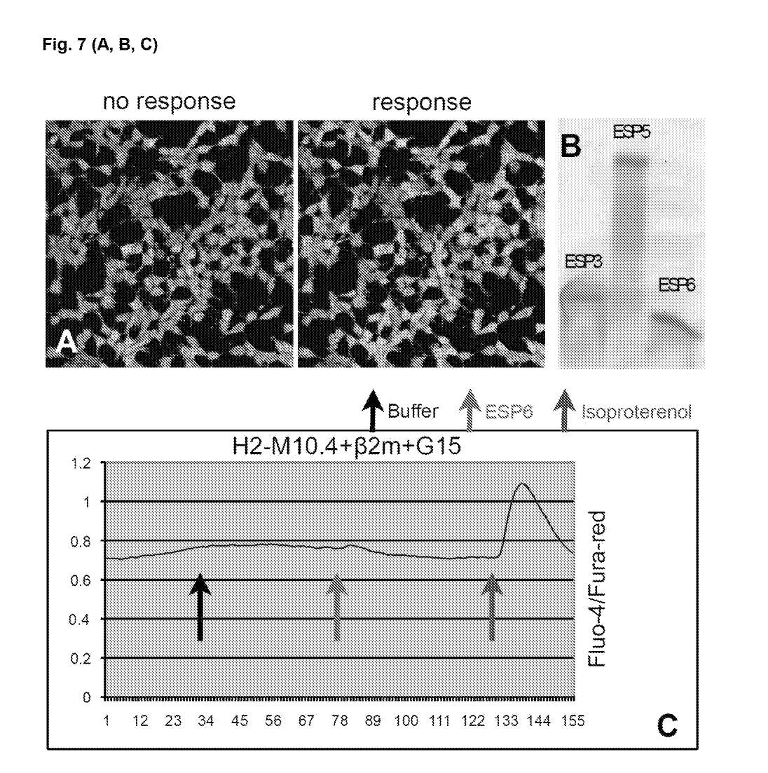

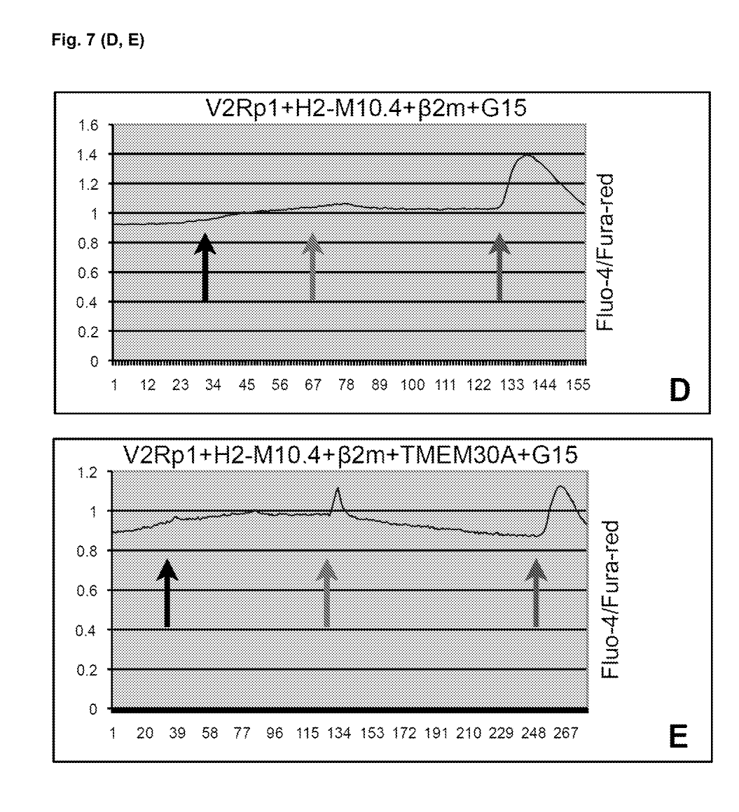

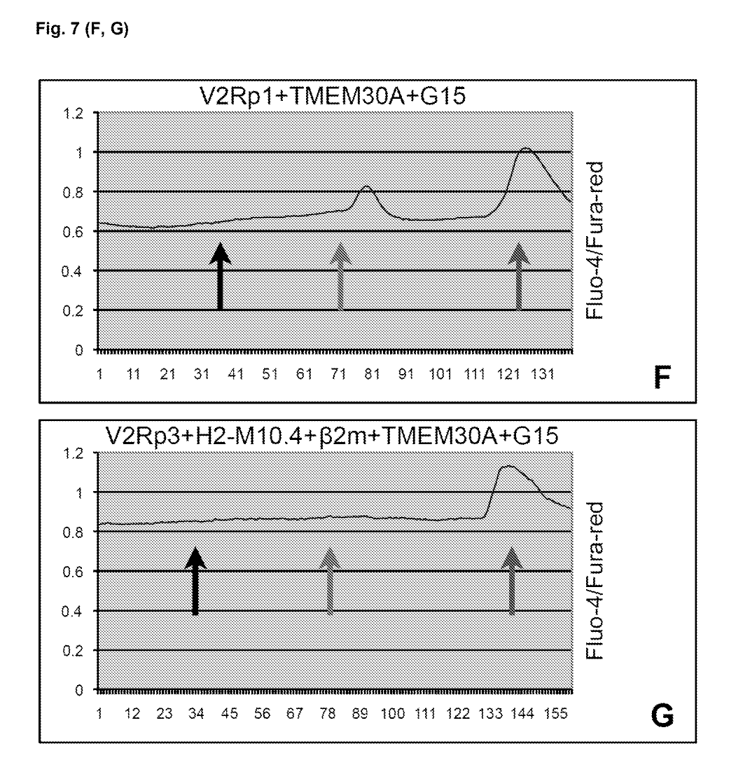

A putative receptor-ligand pair V2Rp1-ESP6 was used as a first step to test whether the V2Rs targeted to the cell surface by Tmem30A are functional. His-tagged ESPs including ESP1, 5, and 6 were expressed in E. coli and the recombinant proteins were purified (e.g., according to ligand protein preparation' above). In order to test whether heterologous V2Rp1 is responsive to recombinant ESP6, ratiometric calcium imaging was performed on cells transfected with Rho-tagged V2Rp1 and Tmem30A, together with G15 that redirects most GPCR activation towards calcium response (e.g., see ‘calcium imaging’ above). As intracellular calcium concentration increased, the Fluo-4 signal (green) intensity increased while Fura-Red signal (red) intensity decreased. One representative experiment is shown (FIG. 7).

As the above data demonstrates, Tmem30A can target and promote the surface expression of GPCRs in heterologous mammalian cell systems. Thus, Tmem30A co-expression can be utilized to improve the surface expression of GPCRs that are typically difficult to express in recombinant and/or heterologous cell systems, including generally, GPCRs, class C GPCRs, chemosensory GPCRs (voneronasal or taste GPCRs) and thereby provide for scalable methods useful in determining functional analysis, ligand selectivity, and agonist/antagonist screening for any known or newly discovered GPCR.

1. A cell line comprising a first polynucleotide sequence encoding a GPCR and a second polynucleotide sequence having at least 80% nucleic acid sequence similarity to SEQ ID NOs: 2, 4, 6, or 8, wherein said second polynucleotide sequence encodes a Tmem30A polypeptide, and wherein GPCR expression is localized to the cell surface.

2. The cell line of claim 1, wherein the recombinant cell further comprises deletion of a calreticulin.

3. The cell line of any of claim 1 or 2, wherein the recombinant cell further expresses a protein selected from REEP, RTP1, and RTP2.

4. The cell line of any of claims 1-3, wherein the GPCR is a class C GPCR.

5. The cell line of claim 4, wherein the GPCR is a vomeronasal receptor or an odorant receptor.

6. A cell line comprising a first polynucleotide sequence encoding a GPCR and a second polynucleotide sequence that encodes a Tmem30A protein having at least 80% sequence similarity to any of SEQ ID NOs: 1, 3, 5, or 7.

7. A recombinant cell comprising a first polynucleotide sequence encoding a GPCR and a second polynucleotide sequence having at least 80% nucleic acid sequence similarity to SEQ ID NOs: 2, 4, 6, or 8, wherein said second polynucleotide sequence encodes a Tmem30A polypeptide, and wherein GPCR expression is localized to the cell surface.

8. The recombinant cell of claim 7, wherein the recombinant cell further comprises deletion of a calreticulin.

9. The recombinant cell of any of claim 7 or 8, wherein the recombinant cell further expresses a protein selected from REEP, RTP1, and RTP2.

10. The recombinant cell of any of claims 7-9, wherein the GPCR is a class C GPCR.

11. The recombinant cell of claim 10, wherein the GPCR is a vomeronasal receptor or an odorant receptor.

12. A recombinant cell comprising a first polynucleotide sequence encoding a GPCR and a second polynucleotide sequence that encodes a Tmem30A protein having at least 80% sequence similarity to any of SEQ ID NOs: 1, 3, 5, or 7.

13. A method for expressing a GPCR in a cell, comprising
(a) providing a cell expressing (i) a GPCR and (ii) a Tmem30A protein having at least 80% sequence similarity to any of SEQ ID NOs: 1, 3, 5, or 7; and
(b) propagating, growing, culturing, or maintaining the cell under conditions effective to promote and/or increase the localization of the GPCR to the cell membrane, cell surface, or combination thereof.

14. A method for identifying a GPCR ligand, comprising:
(a) providing a cell expressing (i) a GPCR and (ii) a Tmem30A protein having at least 80% sequence similarity to any of SEQ ID NOs: 1, 3, 5, or 7;
(b) propagating, growing, culturing, or maintaining the cell under conditions effective to promote and/or increase the localization of the GPCR to the cell membrane, cell surface, or combination thereof;
(c) contacting the cell in culture or in vitro with a candidate GPCR ligand under conditions that allow for binding of the candidate GPCR ligand to the GPCR; and
(d) detecting a signal generated by the binding of the candidate GPCR ligand to the GPCR, wherein the candidate GPCR ligand is identified as a GPCR ligand when a signal is detected.

15. A method for enhancing functional expression of a GPCR in a cell, comprising
(a) providing a cell expressing (i) a GPCR and (ii) a Tmem30A protein having at least 80% sequence similarity to any of SEQ ID NOs: 1, 3, 5, or 7; and
(b) propagating, growing, culturing, or maintaining the cell under conditions effective to promote and/or increase the localization of the GPCR to the cell membrane, cell surface, or combination thereof.

16. A method for increasing localization of a GPCR a cell surface membrane, comprising
(a) providing a cell expressing (i) a GPCR and (ii) a Tmem30A protein having at least 80% sequence similarity to any of SEQ ID NOs: 1, 3, 5, or 7; and
(b) propagating, growing, culturing, or maintaining the cell under conditions effective to promote and/or increase the localization of the GPCR to the cell surface membrane.

17. The method of any one of claims 13-16, wherein the cell further comprises deletion of a calreticulin.

18. The method of any one of claims 13-17, wherein the recombinant cell further expresses a protein selected from REEP, RTP1, and RTP2.

19. The method of any one of claims 13-18, wherein the GPCR is a class C GPCR.

20. The method of claim 19, wherein the GPCR is a vomeronasal receptor or an odorant receptor.


SEQUENCE LISTING (8 sequences)

SEQ ID NO: 1 - protein, 361 aa, Homo sapiens
MAMNYNAKDEVDGGPPCAPGGTAKTRRPDNTAFKQQRLPAWQPILTAGTVLPIFFIIGLIFIPIGIGIFVTSNNIREIEIDYTGTEPSSPCNKCLSPDVTPCFCTINFTLEKSFEGNVFMYYGLSNFYQNHRRYVKSRDDSQLNGDSSALLNPSKECEPYRRNEDKPIAPCGAIANSMFNDTLELFLIGNDSYPIPIALKKKGIAWWTDKNVKFRNPPGGDNLEERFKGTTKPVNWLKPVYMLDSDPDNNGFINEDFIVWMRTAALPTFRKLYRLIERKSDLHPTLPAGRYSLNVTYNYPVHYFDGRKRMILSTISWMGGKNPFLGIAYIAVGSISFLLGVVLLVINHKYRNSSNTADITI

SEQ ID NO: 2 - DNA, 1086 bp, Homo sapiens
atggcgatgaactataacgcgaaggatgaagtggacggtgggcccccgtgtgctccggggggcaccgcgaagactcggagaccggataacacggccttcaaacagcaacggctgccagcttggcagcccatccttacggctggcacggtgctacctattttcttcatcatcggtctcatcttcattcccatcggcattggcatttttgtcacctccaacaacatccgcgagatcgagattgattataccggaacagagccttccagtccctgtaataaatgtttatctccggatgtgacaccttgcttttgtaccattaacttcacactggaaaagtcatttgagggcaacgtgtttatgtattatggactgtctaatttctatcaaaaccatcgtcgttacgtgaaatctcgagatgatagtcaactaaatggagattctagtgctttgcttaatcccagtaaggaatgtgaaccttatcgaagaaatgaagacaaaccaattgctccttgtggagctattgccaacagcatgtttaatgatacattagaattgtttctcattggcaatgattcttatcctatacctatcgctttgaaaaagaaaggtattgcttggtggacagataaaaatgtgaaattcagaaatccccctggaggagacaacctggaagaacgatttaaaggtacaacaaagcctgtgaactggcttaaaccagtttacatgctggattctgacccagataataatggattcataaatgaggattttattgtttggatgcgtactgcagcattacctacttttcgcaagttgtatcgtcttatagaaaggaaaagtgatttacatccaacattaccagctggccgatactctttgaatgtcacatacaattaccctgtacattattttgatggacgaaaacggatgatcttgagcactatttcatggatgggaggaaaaaatccatttttggggattgcttacatcgctgttggatccatctccttccttctgggagttgtactgctagtaattaatcataaatatagaaacagtagtaatacagctgacattaccatttaa

SEQ ID NO: 3 - protein, 364 aa, Mus musculus
MAMNYSAKDEVDGGPAGPPGGAAKTRRPDNTAFKQQRLPAWQPILTAGTVLPTFFIIGLIFIPIGIGIFVTSNNIREIEIDYTGTEPSSPCNKCLSPNVTSCACTINFTLKQSFEGNVFMYYGLSNFYQNHRRYVKSRDDSQLNGDPSALLNPSKECEPYRRNEDRPIAPCGAIANSMFNDTLELYLVANESDPKPIPIPLKKKGIAWWTDKNVKFRNPPGKESLEEKFKDTIKPVNWHKAVYELDPEDESNNGFINEDFIVWMRTAALPTFRKLYRLIERRDDLHPTLPAGQYFLNITYNYPVHSFDGRKRMILSTISWMGGKNPFLGIAYITIGSISFLLGVVLLVINHKYRNSSNTADITI

SEQ ID NO: 4 - DNA, 995 bp, Mus musculus
atggcgatgaactatagcgcgaaggatgaggtggacggcgggcccgcgggtcctcccgggggcgccgccaagacccggaggccggataacacggccttcaaacagcaacggctacccgcctggcagcccatcctcacggccggcacggtgctgcccaccttcttcatcatcggcctcatcttcatccccatcggcatcggcatcttcgtcacctccaacaacatccgtgagatcgagattgattacactggaacagaaccttccagcccctgcaataaatgtttatctccgaatgtgacatcttgtgcttgtaccattaacttcacactgaaacagtcatttgagggcaatgtgtttatgtattatggactgtctaatttctatcaaaatcatcgtcgctacgtgaaatctcgagatgatagccagttaaatggagaccctagtgctttgcttaatccaagtaaggaatgtgaaccttatagaagaaatgaagacagaccaattgcgccatgtggcgctattgccaacagcatgtttaatgatacgttagaattgtatctggttgccaatgaatctgatcccaagcctattccaattcctttgaagaaaaaaggtattgcttggtggacagataaaaatgtgaaattcagaaatccacctggaaaagagagcctcgaagaaaagtttaaagatacaataaagccagtaaactggcataaggcagtatatgagctagaccctgaagatgaaagtaataatggattcataaatgaagactttatagtttggatgcgtactgcagcattacctacttttcgtaagttgtatcgtctcatagagcggagagatgatttacacccaacattaccagctggacagtactttttgaacatcacatacaattaccctgtgcattcctttgatggacggaaacggatgatcttgagcactatttcatggatgggaggaaagaatccatttttgggaattgctta

SEQ ID NO: 5 - protein, 348 aa, Caenorhabditis elegans
MPPRDAVPTSTQVSGIGADGVQTEKVLKNRPKASALRQQKLPAWQPILTATTVIPTVFVIGAIFLPIGVFLFIASDAVSEFTVEYTNCLSPCQLQINLPNAFDGDVFLYYNLENYYQNHRRYVKSRNDQQYLGDLTNVKDCAPFDIDPATKKPIAPCGAIANSIFNDTFTLAHRADTGIVTMVPVTTQGVIWNVDKDRKFKNPPLNDGNLCDAFNNTTKPPNWSKNPCEVGGFENVDFIVWMRTAALPYFKKLWRIVDRTTNPLFSNGLPQGTYILTVENNYPVQSFGGKKEFVISTTSWAGGKNSFLGIAYLVVGSLAIVLGVVFIVIHMKFGHSMNELSNVSEIHH

SEQ ID NO: 6 - DNA, 1047 bp, Caenorhabditis elegans
atgccgccgagagacgccgttccgacgtcgacgcaagtgagtggtattggtgctgacggggttcaaacggaaaaagtgctgaaaaatcggccgaaggcaagcgcccttcgacagcaaaaactccccgcatggcaaccgattctcacagcaaccacagtaattccaacggttttcgtcatcggagccatctttctacccatcggcgtcttcctattcatcgcttccgacgcggtttccgagttcaccgtcgaatacaccaattgcttatcaccgtgccaattgcaaattaatttaccaaatgcgttcgacggagacgtcttcttgtactataatctcgagaattactatcaaaatcatcggcgatacgtgaaaagccggaatgatcagcaatatcttggagatttgacgaacgtgaaggactgtgctccattcgacattgaccctgcgacaaagaagccaattgctccatgcggtgcgattgccaattcgatattcaacgacactttcacgctggctcatcgtgccgacacaggcatcgttaccatggtacccgtgacgactcagggtgtcatctggaatgtggacaaggatcggaaattcaagaatcccccgttgaatgatggcaacctctgtgacgcattcaacaataccacgaaaccaccgaattggagcaaaaatccgtgtgaagttggcggttttgagaacgtggatttcattgtttggatgcgaacggcagctcttccatatttcaaaaagctgtggagaatagtcgaccggaccacgaatccattgttttcaaatggactcccccagggaacttatattttgacagtcgagaataactacccagttcaatcattcggcggaaagaaagagtttgtgatatcgacaacaagttgggctggaggcaagaactcattcctcggaatagcctatctcgtcgtcggatccctggccattgtgctcggcgtagtcttcatcgtgattcacatgaagtttggacattcgatgaacgagttgagcaacgtctcggaaattcatcactga

SEQ ID NO: 7 - protein, 391 aa, Saccharomyces cerevisiae
MVSLFKRGKAPPLTKEGPTSKKPPNTAFRQQRLKAWQPILSPQSVLPLLIFVACIFTPIGIGLIVSATKVQDLTIDYSHCDTKASTTAFEDIPKKYIKYHFKSKVENKPQWRLTENENGEQSCELQFEIPNDIKKSIFIYYKITNFYQNHRRYVQSFDTKQILGEPIKKDDLDTSCSPIRSREDKIIYPCGLIANSMFNDTFSQVLSGIDDTEDYNLTNKHISWSIDRHRFKTTKYNASDIVPPPNWMKKYPDGYTDENLPDIHTWEEFQVWMRTAAFPKFYKLTLKNESASLPKGKYQMNIELNYPISLFGGTKSFVLTTNGAIGGRNMSLGVLYLIVAGLCALFGIIFLVKLIFQPRAMGDHTYLNFDDEENEDYEDVHAENTTLREIL

SEQ ID NO: 8 - DNA, 1176 bp, Saccharomyces cerevisiae
atggtttcattgttcaaaagaggtaaggctccaccgctcacgaaagaaggccccacttctaaaaagcctcctaacacagcgtttagacaacaaaggcttaaggcatggcaaccaatactgtctcctcaaagtgtgcttccgttgttaatattcgttgcatgtatatttactcctattggtattggactcattgtaagcgctactaaggtacaagatctaacaattgattatagtcattgtgatacaaaagcatctacaactgcttttgaagatataccaaagaagtacattaaatatcactttaaaagtaaagttgaaaataaaccacaatggaggctaaccgaaaatgaaaatggcgaacaatcatgcgaactgcagttcgaaatcccaaacgatatcaagaaatccatttttatatattataaaataaccaatttttatcaaaatcatcgcagatatgtccaatcgtttgacacaaagcaaatattaggggagcctatcaaaaaagatgatctggatacaagctgtagtccaataagaagtagggaagacaaaataatatatccctgtgggttgatcgctaattccatgtttaatgatacattttctcaggtgttgagtggtatagatgacacagaagactataatttaactaacaagcatatatcatggagtattgatcgtcacagatttaaaaccaccaagtataatgctagcgatattgttccaccgccaaactggatgaagaagtatcccgatgggtatacagatgaaaatcttcctgatatccatacttgggaagagttccaggtatggatgaggactgcagcctttcccaagttttacaagttgacgttgaaaaatgaatctgcttctttaccgaagggtaaatatcaaatgaacattgagttgaattatccgatttcactctttggtggcacaaaatcatttgtactgactacaaatggagctattggtggtagaaatatgtcactaggcgtactgtacctcatcgttgcagggctttgcgccttatttggcatcatttttttggttaaattaatcttccaaccaagagcgatgggtgatcacacttatttgaattttgatgatgaagaaaacgaggattatgaggatgtacacgcagagaatacaacattgagggaaattttatag